(19) United States
(12) Patent Application Publication
(10) Pub. No.: US 2019/0046429 A1
(43) Pub. Date: Feb. 14, 2019

(54) DERMAL FILLER COMPOSED OF MACROPOROUS CHITOSAN MICROBEADS AND CROSS-LINKED HYALURONIC ACID

(71) Applicant: Prollenium Medical Technologies, Inc., Aurora (CA)

(72) Inventors: Ario Khoshbin, Aurora (CA); Khasha Ighanian, Aurora (CA); Shadi Moghadam, Aurora (CA); Yan Li, Aurora (CA); Hitoshi Masui, Aurora (CA); Stephen J. Kennedy, Aurora (CA); Timothy Lee, Aurora (CA)

(73) Assignee: Prollenium Medical Technologies, Inc., Aurora, ON (CA)

(21) Appl. No.: 16/077,341

(22) PCT Filed: Feb. 9, 2017
(86) PCT No.: PCT/CA2017/050148

§ 371 Date: Aug. 10, 2018

Related U.S. Application Data
(60) Provisional application No. 62/293,358, filed on Feb. 10, 2016.

(51) Int. Cl. A61K 8/73; A61K 8/02; A61L 27/48; A61L 27/58; A61Q 19/08

(52) U.S. Cl. CPC A61K 8/736; A61K 8/735; A61K 8/0283; A61L 27/48; A61L 27/58; A61L 2430/34; A61K 2800/412; A61K 2800/91; A61L 2300/236; A61L 2400/06; A61Q 19/08

(57) ABSTRACT

A biocompatible, degradable dermal filler system is composed of unmodified macroporous chitosan microbeads dispersed uniformly in a continuous phase composed of cross-linked hyaluronic acid gel particles and unmodified hyaluronic acid.

### FIELD OF THE INVENTION

The present invention pertains to biocompatible compositions for soft tissue augmentation, more specifically to a dermal filler containing absorbable chitosan microbeads consisting of pure chitosan, or modified by a chemical crosslinker. The chitosan microbeads are suspended in a matrix of cross-linked hyaluronic gel particles, wherein the microbeads comprise a slowly-resorbing component in an augmentation system designed to provide both short-term and long-term augmentation for the treatment of cosmetic or medical conditions, which require a biocompatible space-occupying substance.

Applications include the treatment of facial wrinkles or folds, or treatment of a wasting medical condition, such as lipoatrophy. In different embodiments of the invention, a pharmaceutical ingredient may be included, for the control of injection pain. Furthermore, the present invention pertains to a process for preparing the chitosan microbeads, and for combining them with cross-linked hyaluronic acid gels, in an augmentation system. The combined system also comprises the formation of a polyelectrolyte complex at the surface of the microbeads, through interaction of HA and chitosan, which is important in regulating the absorption of the microbeads. The macroporous nature of the beads, along with the biocompatibility of chitosan provide a scaffold for the proliferation of fibroblasts and the subsequent deposition of natural collagen.

The present invention also pertains to specific methods of producing the macroporous chitosan microbeads. Both emulsion/solvent evaporation, and emulsion/neutralization methods have been developed. The evaporation method leads to microbeads with large pores, on the scale of the microbead itself, providing an excellent scaffold for cell growth and natural collagen deposition.

### BACKGROUND OF THE INVENTION:

Dermal fillers have been used to offset the effects of aging on the skin, by smoothing soft tissue defects like nasolabial folds and marionette lines as well as more substantial augmentation such as smoothing hollow cheeks resulting from lipoatrophy, or enhancing the fullness of lips. Fillers must be able to satisfy a number of needs, depending on the type of defect which needs to be corrected.

Currently the predominant formulation of dermal fillers is based on chemically-modified Hyaluronic Acid (HA), which has largely supplanted earlier products based on bovine collagen, which suffered from poor durability and the need for an allergy test. Concerns regarding bovine spongiform encephalopathy (BSE) disease also played a role in the declining popularity of the collagen fillers.

Chemical cross-linking of HA is necessary in order to increase the durability of the implant, given that unmodified HA has a half-life in the dermis of only a day or two. Products of this type are Restylane®, Perlane®, Puragen®, the Juvéderm® family, the Esthélis® family, and the Revanesse® family. Notably, all of these fillers are based on cross-linking via the same chemical compound, ButaneDiolDiglycidylEther (BDDE), with differences in the products arising from the details of the manufacturing processes used, including process steps after chemical modification to prepare a sterile, injectable gel, delivered in a pre-filled syringe.

A filler which can also lead to enhanced deposition of natural collagen is very desirable, as the effects would be long-lived and completely natural. There is also a need for a type of filler, which can provide significant enhancement, with long duration as well as natural collagen deposition. This should be accomplished without inducing an ongoing inflammatory reaction. To accomplish this, a delicate balance is required between the rate of absorption of the implant and stimulation of the skin to produce collagen, avoiding overstimulation leading to inflammation.

The inclusion of solid, biocompatible microbeads in a biocompatible carrier has been one approach to meeting this need for long duration and natural collagen deposition. The carrier is usually absorbed in a relatively short period. However, it has proven to be difficult to achieve the correct balance, and these more-permanent fillers have had issues regarding excessive tissue reaction after implantation or too-rapid absorption. These treatment can also be difficult to reverse, if it is deemed necessary, by injection of natural enzymes. This can be done with the HA-based fillers (hyaluronidase). An ideal combination of durability and stimulation of collagen deposition, with low inflammation has yet to be achieved.

Some materials that have been used for these microbeads are polymethylmethacrylate (PMMA), described in U.S. Pat. No. 5,344,452, polylactides (polylactic acid or PLA), described in U.S. Pat. No. 6,716,251, and calcium hydroxylapatite, described in U.S. Pat. No. 7,060,287. Recent patents describing the potential use of polycaprolactone or polydioxanone microbeads, are U.S. Pat. Nos. 7,964,211, and 9,119,902. All of these biomaterials have been used previously in other medical applications: PMMA in intraocular lenses and bone cement, PLA in bone pins and screws, polycaprolactone in absorbable sutures and drug delivery devices, and hydroxylapatite as a bone filler and contrast agent. These materials were then adapted for use in a dermal filler by forming microbeads from the raw material. Durability of these fillers ranges from months, to several years to permanent.

Typically these microbeads are sized between 40 and 150 μm. If the spheres are too small they can elicit a reaction from macrophages, which will attempt to engulf the particles. If the spheres are too large, they cannot be injected with the fine needles that are used in dermal filling procedures, and they may be palpable under the skin.

Chitosan is a naturally-occurring biopolymer, a linear polysaccharide composed of randomly distributed β-(1-4)-linked D-glucosamine and N-acetyl-D-glucosamine units. A related form is chitin, which is simply a linear chain of β-(1-4)-linked N-acetyl-D-glucosamine, and from which chitosan can be derived. Chitin is the primary component of arthropod exoskeletons and is the second most abundant naturally-occurring biopolymer after cellulose. Chitin is also the source for most commercial chitosan.

Both chitin and chitosan have a number of industrial uses, e.g. food processing and preservation, waste-water treatment, and chromatography. In the medical field chitosan has drawn attention due primarily to its biocompatibility, biodegradability, and antimicrobial properties. Chitosan has been extensively studied for tissue engineering and drug delivery applications. The ability to form microspheres as well as polyelectrolyte complexes with anionic biopolymers like alginate, carboxymethylcellulose, and hyaluronic acid is important in some of these proposed applications. The mucoadhesive properties of chitosan have also been studied for use in nasal and ocular drug delivery. The ability of chitosan and chitin to speed the wound healing process has been known for decades and commercial products have been marketed. The antimicrobial property as well as the promotion of healing are beneficial in a dermal filler application as well.

Chitin and chitosan have a cellulose-like molecular structure. Chitin, due to the stability of its crystalline form, is insoluble in water. The random arrangement of acetylated and deacetylated units along the chitosan chain and the possibility for protonation of the glucosamine units, means that chitosan is soluble in weakly acidic aqueous solutions, where it exists in the form of a polycation. The precise pH at which chitosan dissolves depends on the molecular weight, the degree of deactylation (proportion of glucosamine units), and the degree of randomness of the arrangement of the two units along the chain. Chitosan with a partial block-like structure is produced in some processes, and this material is less soluble in water.

A commonly noted drawback of chitosan is that it is generally insoluble at a pH above approximately 6.5, which is slightly lower than physiological pH. This has hindered its adoption in medicine for some applications. Another issue has been the difficulty in sourcing high-quality raw material. A great deal of research has been directed towards methods of increasing the aqueous solubility of chitosan by derivitization, choice of counterion, or by meticulous control of molecular weight, degree of deacetylation, and the distribution of base units along the linear chain. Generally, the goal has been to develop hydrogels that could be used, for example, in drug delivery or tissue engineering.

### Objects of the Invention:

The objects of the present invention are to provide a composition for dermal filler applications that can be injected with a fine-gauge needle and provide 1) an immediate augmentation effect from a proven HA-based composition, which additionally includes a chitosan microbead component, 2) provide long-lasting augmentation from the chitosan microbeads, as the HA gel is absorbed, 3) provide for the deposition of natural collagen, around and inside the microbeads, as they are slowly absorbed, with 4) no excessive inflammatory reaction, or formation of granulomas.

These objectives constitute ideal properties for a long-lasting tissue augmentation product, in particular for a cosmetic dermal filler, where longer duration of the correction is desired.

All four objectives have been demonstrated for our composition in a year-long rat implantation study, which included three implementations of our invention, with high-density and low-density macroporous unmodified chitosan microbeads as well as a formulation containing chemically-crosslinked chitosan microbeads.

In the present invention, the drawback that chitosan does not dissolve readily in tissue and remains a solid after implantation, becomes an advantage. Simple, unmodified chitosan microbeads can be used in a filler application, and no additional chemical stabilization of this biocompatible polysaccharide is necessary. However, the borderline insolubility of chitosan at physiological pH means that the material is not inert, and will be slowly degraded at the implantation site. The monomeric components of chitosan are N-acetyl glucosamine and glucosamine. Both of these monosaccharides are naturally occurring in the human body, and are necessarily biocompatible. Our primary invention comprises the use of unmodified chitosan microbeads combined with cross-linked hyaluronic acid gel in a dermal filler. The invention includes the specific process by which the microbeads are formed, leading to a macroporous structure, as well as the discovery that an HA-Chitosan polyelectrolyte complex forms at the surface of the microbeads in our formulation and significantly enhances the durability of the microbeads.

We also claim an additional embodiment of our invention, which uses a chemical cross-linker to further stabilize the chitosan microbead component and extend the duration of the implant, for applications where a more permanent augmentation is desired. We claim the use of unmodified microbeads from either of our productions processes, and adding the step of chemical cross-linking between sites on the chitosan polymer chain in the previously-formed microbead.

Given the existence of primary amines (glucosamine) along the chitosan chain, there are many methods that can be employed. Amines will react with a wide variety of bifunctional cross-linkers. For one skilled in the art, these include biisothiocyanates forming isothiourea bonds, biisocyanates forming isourea bonds, biazides forming amide bonds, dialdehydes forming secondary amines after reduction, dicarboxylic acids, esterified by N-hydroxysuccinimide (NHS), or sulfo-NHS, forming amide bonds, dicarboxylic acids in combination with carbodiimides forming amide bonds, and bisepoxides forming secondary amine bonds.

In the present invention, bisepoxides are preferred, and BDDE is most preferred, due to its widespread use in stabilizing hyaluronic acid for dermal filler applications and the established safety profile it has in this product area.

### SUMMARY OF THE INVENTION

The present invention is directed to a dermal filler comprising a combination of biocompatible, absorbable, macroporous chitosan microbeads dispersed in a gel particle phase of cross-linked hyaluronic acid. Also described are methods for making such microbeads as well as standard methods for producing the cross-linked HA gel phase.

The production of chitosan microbeads (or microspheres) has been considered by a number of researchers. Standard techniques have been employed. Generally droplets of a chitosan solution are formed, and hardened into microbeads by different methods.

Aqueous chitosan solution droplets can be formed and hardened into microbeads in a spray dryer. The solution is forced through a nozzle or expelled by a spinning disk, into a heated gas phase, wherein the water from the droplet evaporates and a microbead is formed. Difficulties arise in controlling the size and shape of the microbeads.

A simple method to produce chitosan beads is by extrusion through nozzles or needles of an acidic chitosan solution into an alkaline solution, called ionotropic gelation, or more simply, neutralization. Chitosan is a polycation, so chitosan solution droplets can also be extruded into a solution of a polyanion like triphosphate, for physical cross-linking. After extrusion the increase in the pH or the presence of the polyanion lowers the solubility of the chitosan, initially forming a gel particle from the droplet and finally solid beads. Generally in these methods the beads are relatively large and shapes are often poorly controlled. Methods for forming small, uniform-sized microbeads, such as injection of droplets from microfluidic devices offer good control of dispersity, but are impractical due to the low production rates that are achievable, particularly if the goal is to produce small droplets, with microbeads with diameters on the order of 100 μm.

A more practical means of producing large numbers of small microbeads is to produce the droplets by emulsification of a chitosan solution in a non-aqueous phase. The hardening of the droplets into microbeads can be accomplished by changes in pH or the addition of chemical or physical cross-linking agents. In our initial experience, addition of an alkaline solution to the emulsion resulted in rapid gelation at the surface, with low-density microbeads, that collapse on drying with poor control over the final shape. After drying, the microbeads were reduced in size and did not absorb water well, due to a high degree of crystallinity in the solid. However, a modified emulsion/neutralization method based on discoveries we made during the development of our emulsion/solvent evaporation method was more successful and is described below as Method 2.

Another approach based on emulsification is to choose an ‘oil phase’ with a non-zero solubility for water, but no solubility for chitosan. One example of this method, described by Baimark and Srisuwan (2013) is to use ethyl acetate as the ‘oil’ in a W/O emulsion of a chitosan solution. After the emulsion is formed water is drawn out of the droplets into the organic phase in which it is partially soluble, and eventually solid microbeads can be obtained. The drawback to this approach is the need to use very large amounts of the organic solvent. Some fraction of water must dissolve into the organic phase in this approach, but the concentration is typically very low for a system that must also form two distinct phases. As a result this method requires large amounts of organic solvent relative to the mass of microbeads that can be produced. Given these limitations solvent diffusion was not pursued as a suitable commercial method, for manufacture of our microbeads.

Method 1: Another emulsion-based method which can produce small microbeads with acceptable control over the size distribution are emulsion/solvent-evaporation methods. In this approach a stable emulsion is produced with controlled-size droplets. After an appropriate change in conditions, such as elevating the temperature, a loss of solvent occurs in the droplets, until a solid microbead is formed. This method requires evaporation of water through an immiscible non-aqueous phase. This can be done slowly, leading to a more spherical shape and smooth surface structure for the microbeads. This is the favored approach we employ in our invention to form the unmodified chitosan microbeads, which is also the source material for the cross-linked version of our microbeads.

The emulsion from which the microbeads are produced is formed in two stages. First, the initial emulsion is obtained by homogenizing an aqueous acidic chitosan solution in an oil phase in the presence of emulsifier, to form an initial W/O emulsion. This primary emulsion is then stabilized by dilution with additional oil to form the secondary emulsion. Afterwards the chitosan microbeads are formed from the droplets in the secondary emulsion by diffusion of water through the oil phase and evaporation at the surface and by agglomeration of the primary particles into the microbeads.

A unique aspect of our process is the formation of an Oil-in-Water-in-Oil, or 0/W/0 emulsion, in the primary emulsion, due to the high W/O ratio possible with the choice of castor oil for the primary emulsion. During the drying phase in the secondary emulsion the primary gel particles aggregate to form the final microbead, but these carry with them a portion of the castor oil phase, which coalesces into large oil droplets in the microbead as it forms (FIG. 2). After the washing step removes the oil, the resulting voids are the source of the large pores in the final microbead.

It is the formation of this special phase that results in the large macroporous structure of our beads after removal of the entrapped oil, and is an important aspect of the invention. In the final step, the microbeads are washed with an organic solvent, removing the oil phase, and leaving a macroporous structure in the resulting dried microbead. The macroporosity is a key feature, which assists in controlling the degradation of the microbeads and allows for the ingress and proliferation of fibroblasts leading to slow replacement of the microbead by natural collagen deposition, after implantation in tissue. Evidence from our rat implantation study supports this conclusion as shown in FIGS. 8 and 9.

Some of the process parameters that affect the final microbead product are the composition of the aqueous and oil phases, the molecular weight of the chitosan and its degree of deacetylation, the mixing geometry, speed, and time, the water/oil phase ratio, the evaporation temperature and even the external conditions of surface to volume ratio, humidity and air flow. These can affect not only the size and shape of the microbeads, but the surface smoothness and porosity. Conditions employed in our invention are described and claimed as part of the invention in the detailed description and examples below.

As noted, the microbeads are cleaned, neutralized, and dried, obtaining highly purified macroporous microbeads appropriate for inclusion in an injectable product. The ‘near solubility’ of the microbead is demonstrated by the observation that it swells significantly, by approximately 50%, but does not dissolve when equilibrated in phosphate-buffered saline at a pH of 7.0. The microbeads prepared by our process flow well, and can be uniformly dispersed into the cross-linked HA gel particle phase at the desired concentration, without damage. After formulation the microbeads produced in our process can also withstand automatic filling into syringes and terminal sterilization by moist heat in an autoclave. The method is described in detail below as Method 1.

Method 2: During the development of the solvent evaporation method the discoveries we made concerning the properties of different oils allowed us to develop an improved approach to the emulsion/neutralization method. Briefly a chitosan solution can be dispersed in castor oil, with or without the addition of an emulsifier.

In previous attempts by us to use the neutralization method, aqueous bases such as a sodium hydroxide solution were employed. This formed a second water phase, with droplets of sodium hydroxide solution. As these came in contact with the chitosan solution the pH would be lowered and the droplets hardened. However, this produced a very irregular distribution of microbeads, with many large agglomerates.

In the method described here, the droplets are neutralized by addition of a base with significant solubility in the castor oil phase. This is an important discovery, as we find that it leads to microbeads with essentially spherical shapes and good control over the size distribution. The fact that the base can reach the aqueous droplets of chitosan solution by diffusion through the oil phase is the key to this improvement. We find that lowering the pH in this way causes solidification in as little as 20 minutes.

Dilution with solvents miscible with both water and castor oil, permits isolation of these microbeads by filtration or sedimentation. This method also produces porous chitosan microbeads, with a somewhat broader size distribution than the solvent evaporation method. This alternative method for production of chitosan microbeads is described in detail below as Method 2.

### DETAILED DESCRIPTION OF THE INVENTION

### Chitosan Bead Formation—Method 1: Emulsion/Solvent Evaporation

Many factors affect the final state of the chitosan microbeads formed in our process: the molecular weight and degree of deacetylation of the chitosan; the type of acid and the concentration used in the chitosan solution; the chitosan concentration; the composition and resulting hydrophobicity and viscosity of the oil phase; the type and concentration of the emulsifier; the O/W ratio in the primary and secondary emulsions, the mixing apparatus used; and the evaporation conditions, including temperature and geometry.

Chitosan Raw Material:

Choice of raw material is important in the production of high quality chitosan microbeads. As noted above, two factors are important in determining the physical properties of chitosan, the molecular weight and the degree of deacetylation. The distribution along the chain can also be important but this is not determined by suppliers.

In our method, to be described here in detail, we have discovered that increasing molecular weight tends to produce more spherical microbeads, with a smoother surface, and a narrower size distribution. We have also determined that the degree of deacetylation mainly affects the crystallinity of the microbeads, which in turn affects the degree of swelling when the dried beads are rehydrated. The crystallinity will also affect the rate at which degradation will occur in-vivo.

Many suppliers provide only a wide specification range for molecular weight and degree of deacetylation. We have determined that, in our method, molecular weights from 100 to 2000 kDa can be used, more preferably from 300 to 700 kDa. A degree of deacetylation from 65 to 95% can be used, more preferably, from 80 to 90%.

Chitosan Solution:

At low pH amine groups on chitosan are protonated, increasing the solubility in aqueous solution. Chitosan can be dissolved in a variety of organic acids such as acetic acid, formic acid, adipic acid, ascorbic acid and lactic acid, or dilute inorganic acids such as hydrochloric acid or phosphoric acid. Any of these can be used in our method and are included in the invention. Preferably the solution is prepared with either acetic acid or dilute hydrochloric acid, and most preferably with acetic acid.

The molecular weight, the degree of deacetylation, the concentration and the pH all affect the physical properties of the chitosan solution, most importantly the viscosity. This affects the size of the droplets, with other conditions held constant. The concentration and size of the droplets determines the size and porosity of the resulting chitosan microbeads. These factors can all be adjusted to produce a range of microbead sizes, swelling characteristics, and final chitosan density in the swelled microbeads.

For example: chitosan with a molecular weight specification of between 140 kDa and 220 kDa at a concentration of 4% dissolved in 5% AcOH has a viscosity of 30.28 Pa·s, while a solution with a concentration of 4.44% in 10% AcOH has a similar viscosity of 31.25 Pa·s. We have discovered that those solutions with the same viscosity, produce the same particle size in our method, however with different densities. This is an example of how various inputs in our process can be used to control the final properties of the microbeads.

Acceptable concentrations of chitosan in our method depend on the molecular weight and on the concentration of acid but are between 1% and 5%, preferably between 2% and 3%. Hydrochloric acid concentrations can be used between 0.1N and 0.2N, most preferably between 0.16N and 0.18N. Lactic acid concentrations can be used between 1 and 10%, most preferably between 2% and 3%. Acetic acid concentrations can range between 2% and 10%, preferably between 4% and 6% and most preferably at 5%.

Oil Phase Composition:

A wide variety of non-toxic oils can be used as the continuous phase in the primary emulsion. For the highest levels of safety, preferred oils are those listed in the FDA Inactive ingredients guide for intramuscular, intravenous, or intradermal injection. These oils have been used in the development of various drug products, or drug delivery systems. These include vegetable oils, such as corn oil, soybean oil, canola oil, castor oil, sesame oil, peanut oil and almond oil, or light mineral oil and mineral oil. The oil is not part of the final formulation of the filler system of course, but restricting the production process to the use of these oils, adds to the assurance of safety for the final product.

Three important parameters for the oil phase that pertain to our method are the viscosity, the polarity of the oil, and the interfacial tension between the oil and aqueous phase. The aqueous chitosan solution is viscous and we have discovered that the more closely the viscosity of the oil phase matches the chitosan solution, the more uniform the size distribution of the microbeads. High interfacial tension is obtained with hydrophobic, non-polar oils like mineral oil, and this tends to produce microbeads with a smooth surface and spherical shape. However, water evaporation from the droplets, which is necessary to form the microbeads, is impractically slow when the oil continuous phase is very hydrophobic, as there is extremely limited solubility of water in these oils.

In addition, the low viscosity of the mineral oils relative to the chitosan solution, tends to produce microbeads with a wide size distribution. A wide distribution was also noted with some of the vegetable oils, such as corn oil, also due to relatively low viscosity. The low viscosity of these oils also tended to produce less stable emulsions due to increased rates of droplet coalescence, allowing less time for transfer to the secondary emulsion.

Castor oil was found to be an excellent choice on the basis of its high polarity (due to the hydroxyl group on ricinoleic acid), and its high viscosity, which is a good match to the viscosity of the chitosan solutions in our method. The result is good uniformity in the size distribution of droplets in the primary emulsion. The high viscosity also allows for a high W/O ratio (up to 9/10), important for commercial rates of production. We also discovered that these high ratios of W/O led to the formation of the O/W/O multiple emulsion, critical to the formation of the macroporous structure of the final microbeads, shown for example in FIGS. 8 and 9. The dependence of the formation of the multiple emulsion and the subsequent macroporous structure of the beads is not obvious, and is an important part of the invention described here.

Water is slightly soluble in castor oil, due to its polarity, which allows for an acceptable evaporation rate, with water moving from the droplets to the oil and then evaporating at the surface. However that same polarity and slight miscibility with water, leads to a low value for the interfacial tension, leading in turn to poor sphericity and smoothness for the microbeads.

Castor oil proved to be an ideal oil for the primary emulsion. However the issues noted above indicated that a mixture of castor oil and mineral oil if used for the oil phase in the secondary emulsion, might lead to an improved local structure for the microbeads. It is in the secondary emulsion where the evaporation/hardening of the droplets and aggregation into the chitosan microbeads occurs. However these two oils, due to their difference in polarity are not miscible. However, we have made the discovery that oils with an intermediate polarity from the list of those oils approved for injection by the US FDA, can act as solubilizing agents between castor oil and light mineral oil. The preferred oil for this purpose is corn oil.

The coexistence curve for the three component system, castor oil, light mineral oil, and corn oil, has been mapped on a ternary phase diagram at two temperatures. This is shown in FIG. 1. We have found that a single-phase region exists, dependent on the temperature, wherein the combination of oils is miscible and this combination can be used as the continuous phase in our secondary emulsion. So, we have discovered a means, using oils that we consider acceptable, for adjusting important properties of the oil phase, to optimize the production rate and quality of the chitosan microbeads. The combination of mineral and castor oil by the addition of a third oil (corn oil in this particular embodiment) is an important part of our process and the invention described herein.

A range of compositions for the secondary emulsion oil phase can be used with components of castor oil, corn oil and light mineral oil. Based on a nunber of designed experiments, our preferred composition ratio is 10/20/20 for castor oil/corn oil/light mineral oil.

Emulsifier:

A variety of biologically compatible emulsifiers (FDA-approved for either injection or transdermal application) can be used including the two families of sorbitol derivatives, the hydrophobic Span® family, in particular Sorbitan Monopalmitate and Sorbitan Monooleate, and the hydrophilic Tween® family, particularly PEG-20 Sorbitan Isostearate. Castor oil derivatives such as PEG-40 Castor Oil, PEG-60 Hydrogenated Castor Oil, and Polyoxyl 35 Castor Oil can also be used, as well as the Glyceryl derivatives, Glyceryl Palmitostearate, Glyceryl Oleate, Glyceryl Trioleate, and Glyceryl Laurate, as well as the Poloxamer family of nonionic emulsifiers, in particular Poloxamer 188. Hydrogenated Soybean Lecithin can also be used. Most preferred is the natural emulsifier, Lecithin.

In the primary emulsification of the chitosan solution, a range of concentrations of lecithin can be used, from 1% to 5%. Most preferred is 2% lecithin in castor oil.

Primary Emulsification Process:

In our method, emulsification can be carried out in a simple mixing system. There is no necessity for turbine-style mixers, high-pressure homogenizers (Manton-Gaulin, Microfluider®), or colloid mills, although these could potentially be used.

We have however observed that mixing speed and duration can influence the particle size, within a certain range. For example, a sample prepared at 450 rpm for 1.5 minutes of mixing time shows a similar mean particle size (65.8 μm) as another sample, which was prepared at 400 rpm and 2 min (63.7 μm), holding other parameters constant.

As noted, we have also made an important discovery, that by using a sufficiently high W/O ratio in the primary emulsion with castor oil, that droplets of oil are also incorporated into the aqueous chitosan phase. We have at that stage an O/W/O multiple emulsion. After aggregation of primary gel particles to form solid microbeads, the coalesced oil droplets are later removed in the final washing step, leaving behind macroporous chitosan microbeads. The pore structure assists in controlling the degradation of the microbeads and allows for the ingress and proliferation of fibroblasts leading to slow replacement of the microbead by natural collagen deposition.

Secondary Emulsion and Evaporation:

A secondary emulsion is prepared by dilution of the primary emulsion in an excess of the oil phase used in the primary emulsion. By forming a dilute suspension of the aqueous chitosan solution droplets in an excess of the oil phase, we control the stability for the evaporation/hardening step, by reducing the frequency of droplet-droplet collisions. Under the conditions, wherein lecithin is used at 2% in castor oil for the primary emulsion and the W/O ratio is 0.9, we have discovered that no additional lecithin is required in the oil fraction of the secondary emulsion for the preferred final composition of 10/20/20 for castor oil/corn oil/light mineral oil, in order to maintain the stability of the droplets during evaporation. We have also discovered that continued mild stirring of the secondary emulsion is the best condition to maintain stability during the evaporation process.

The evaporation temperature, along with the geometry of the hardening tank, and the external conditions of air flow and humidity all affect the evaporation rate. Temperature is important in several respects. Although higher temperatures shorten the drying/hardening time, higher temperatures can also destabilize the emulsion. We have found that temperatures for the secondary emulsion between 20° C. and 40° C. can be used in the evaporation process, more preferably between 20° C. and 30° C., and most preferably between 26° C. and 28° C. In particular these conditions are ideal for the preferred oil composition of 10/20/20, castor oil/corn oil/light mineral oil.

As noted in the brief description the formation of the microbeads in the secondary emulsion is a complex process. Incorporated into the primary aqueous droplets are some droplets of the castor oil phase. As evaporation begins and the droplets develop a gel-like nature there is also some aggregation of the gel particles, and finally the formation of solid microbeads, which still incorporate oil. The contraction of the microbead on drying leads to the existence of relatively large pores in the structure of the bead. The removal of these pores in the washing process leads to the final macroporous structure of the chitosan microbeads, evident in FIGS. 8 and 9.

Washing and Neutralization:

Washing: The secondary emulsion turns clear after continuous stirring for 10-24 hours, indicating the microbeads are solidified. The oil phase containing microspheres is then transferred to a centrifugation tube and centrifuged at 1000 G for 1 min, the supernatant oil is decanted, and the microspheres at the bottom are then washed twice with ethyl acetate and twice with n-hexane by vortex mixing, followed by centrifugation, to remove the residual oil. Trace amounts of lecithin are removed by washing twice with ethanol, and then the clean beads are allowed to dry in air.

Neutralization: Chitosan beads as obtained were first soaked in saturated Na₂CO₃ solution for 10 minutes, then washed with DI water several times to produce the protonated form of chitosan. They were further subjected to washing with PBS, until a neutral pH was achieved. After the removal of surface salt by rinsing with DI H₂O, the beads were dehydrated with ethanol and air dried. The final neutral beads were stored in sealed vial at room temperature prior to use.

Final Mass Density:

After swelling in phosphate-buffered saline, the beads swell by approximately 50%, and the final mass concentration of chitosan in the microbeads ranges from 8% to 20% but preferably between 10% and 16%. For unmodified chitosan microbeads, the densities, 9.9% and 16.2%, were used in the rat implant study. The details of the process for preparing these microbeads are described in Examples 1, and 2. The results of the implant study are discussed below.

Size Distribution:

Typical size distributions are shown in FIG. 1, wherein the mean size is approximately 95 μm with a standard deviation of 20 μm.

Cross-Linking of Chitosan Microbeads:

Our invention demonstrates the feasibility and desirability of using umodified chitosan microbeads in a dermal filler system in combination with cross-linked hyaluronic acid gel particles. However, we have also discovered a simple method, using BDDE, a chemical cross-linker well-known in the art of HA-based dermal fillers, to produce chemically cross-linked chitosan microbeads, with the same macroporous structure as the unmodified beads described above. This is accomplished by using microbeads from Method 1. The cross-linking occurs under basic conditions, so the microbead structure is maintained. An example of this process is provided in Example 3.

Although the microbeads are not solubilized undert the basic conditions used during the cross-linking, the reaction still occurs. This discovery is also part of the invention described herein. The existence of chemical cross-links in these modified microbeads is demonstrated by a lack of solubility in 5% acetic acid solution, which will readily dissolve the unmodified microbeads used as source material in the cross-linking reaction. Cross-linked microbeads with a density of 14.7% were used in the rat implant study and those results are also discussed below.

Chitosan Bead Formation—Method 2: Emulsion/Neutralization Hardening

Given the unique characteristics we had discovered for a W/O emulsion of chitosan solution in castor oil, we examined again the emulsion/neutralization process for forming microbeads from an emulsion of an acidic chitosan solution dispersed in a castor oil continuous phase. We were surprised that these results were superior to those achieved using other emulsion systems. We also made discoveries regarding the types of base that must be used for neutralization, which were dependent on their solubility in the castor oil phase.

Basic steps in this method involve the dispersion of an aqueous acidic chitosan solution in an oil phase, most preferably castor oil, followed by the addition of a base to precipitate the solution droplets into a gel/solid phase, followed by dilution with an oil-miscible low-viscosity organic solvent to allow for separation of the microbeads by centrifugation or filtration. The collected microbeads are then washed and dried, or washed and suspended in phosphate-buffered saline.

Surprisingly we discovered that chitosan microbeads could be produced from a primary emulsion in castor oil, either with an emulsifier as in our solvent evaporation method, or in the absence of an emulsifier, depending on the desired size of the microbeads. We discovered that castor oil intrinsically acts as a weak emulsifier, due to the hydroxyl group on the aliphatic chain of ricinoleic acid, the primary fatty acid in castor oil. Surprisingly, the stability of the dispersion is adequate, given that our neutralization method is very rapid. An emulsifier, such as lecithin does have the effect of reducing the droplet size, if other conditions such as W/O ratio, mixing apparatus, and temperature remain constant, but is not essential.

We discovered that ordinary aqueous bases like sodium hydroxide solution produced irregular, poorly-controlled microbeads, often with large agglomerates. We subsequently discovered that if the base has significant solubility in the castor oil phase, superior results, with the formation of spherical beads with adequate size control are possible. This is true even if an aqueous solution of the base is used, as long as the base itself has solubility in the castor oil.

A variety of bases can be used for neutralization as long as there is some solubility in the castor oil phase, or other oil phase used. Amine bases can be used, such as the aliphatic primary amines, methylamine, ethylamine, propylamine, isopropylamine, and other members of this family; secondary amines such as dimethylamine, and tertiary amines such as triethylamine can also be used. Amino alcohols such as ethanolamine, triethanolamine, and tris(hydroxymethyl)aminomethane are also effective neutralizers. Ammonia gas, NH₃ can be used to neutralize the droplets by bubbling the gas through the emulsion, or more conveniently, a concentrated NH₄OH solution can be added to the emulsion. As noted above, although the aqueous NH₄OH solution forms a separate dispersed phase in the emulsion, we have discovered that neutralization of the chitosan droplets occurs at a good rate by diffusion of ammonia from the ammonium hydroxide solution through the oil phase to the droplets. The microbeads which are formed in this process are also porous with a density comparable to those obtained in the solvent evaporation method described previously.

After neutralization the microbeads can be separated from the oil phase by the addition of a oil-compatible diluent, to lower the viscosity of the continuous phase and allow for separation of the microbeads from the continuous phase, either by filtration or centrifugation. A variety of common organic solvents can be used as a diluent, if they are miscible with castor oil. Particularly useful are solvents, which are also miscible with water, and relatively volatile, to make removal of the solvent simple. Some examples are acetone or acetonitrile, or alcohols such as methanol, ethanol, or propanol. Most preferred is ethanol for reasons of toxicity as well as ease of removal.

The same solvent can then be used to wash the microbeads and remove any residual oil. Resuspending the microbeads in PBS, tends to maintain the spherical shape, until needed for incorporation into the filler, and we have found it superior to complete drying of the microbeads, although we have also followed this procedure and successfully obtained microbeads.

Other discoveries disclosed herein, that are part of the invention is the observation that in this simple method, chitosan solution must be added to the oil, either drop wise or by injection, while stirring, to avoid excessive encapsulation of oil in the chitosan microbeads and disruption of the microbead structure; and the emulsion must also be fully neutralized by the base before workup, to avoid agglomeration and fragmentation of the microbeads.

It was discovered that if the viscosity of the chitosan solution was 2 or 3 times that of castor oil, but not higher, microbeads of the desired size, around 100 μm could be produced. The viscosity of the chitosan solution depends on molecular weight and concentration. A 2.5% solution of a low-molecular weight chitosan of 300 kDa produced good results. It was also discovered that these microbeads could be dried and reswelled without undergoing excessive deformation. The conditions used are described in Example 4.

These microbeads also have a macroporous structure. This is indicated from the bead density and also the partial transparency of the microbead.

Examples of Microbead Production Processes:

Method 1: Emulsion/Solvent Evaporation

### EXAMPLE 1

### Low-Density Microbeads

Briefly, 200 mg of chitosan was completely dissolved in 11 ml of 0.1N HCl via manual mixing between 2 syringes connected with a luer-to-luer adapter, the solution was allowed to stand overnight to get rid of the bubbles. Meanwhile, 2% lecithin was dissolved in castor oil by heating at 120° C. for 0.5 hour under magnetic stirring. Afterwards, the aqueous phase (8 g) was added into the oil phase (10 g) in a 50 ml beaker, the emulsification was performed at 400 rpm for 1.5 minutes with an overhead stirrer utilizing an anchor propeller.

Subsequently, the primary emulsion was quickly poured into a large amount of an oil phase consisting of 20 g of light mineral oil and 20 g of corn oil, under constant magnetic stirring at 500 rpm. In order to allow the microbeads to solidify, the stirring was continued for 18 hours at 28° C.

The oil phase containing microspheres was centrifuged at 1,000×g for 1 min. The supernatant oil was decanted, and the micromicrobeads at the bottom were then washed 2 times with ethyl acetate, and 2 times with ethanol by centrifugation to remove the oil and excess emulsifier. They were then neutralized by soaking in 5 M Na2CO3 solution for 10 minutes, washed with water to remove the salt on the surface, then they were washed with ethanol, and air-dried.

The resultant chitosan microbeads can be stored under room condition in the dry state, the content of chitosan in the wet microbeads is around 10%.

### EXAMPLE 2

### High-Density Microbeads

Dissolve 300 mg of chitosan in 11 ml of 0.1N HCl via manual mixing between 2 syringes connected with a luer-to-luer adapter, the microbeads were prepared using the same procedure in Example 1 except the emulsification speed was increased to 450 rpm. The obtained micromicrobeads have a higher solid content (˜15%).

### EXAMPLE 3

### BDDE Cross-Linked Medium-Density Microbeads

Preparation of ‘medium-density’ microbeads:

Dissolve 230 mg of chitosan in 11 ml of 0.1N HCl via overhead stirring, the microbeads were prepared using the same procedure in Example 1 except the emulsification speed was decreased to 350 rpm for 2 minutes. The obtained microbeads have a medium solid content (˜14%) in the swelling state.

Cross-linking

First weigh around 280 mg of plain chitosan beads into 16 ml jacketed beaker, then add 10 ml of 1% NaOH into this beaker, suspend the beads under mild magnetic stirring with a thin stirrer, keep continuous mixing for 20 min. Second, 100 μl of BDDE was added into the beads suspension, the cross-linking reaction was allowed to proceed at 50° C. for 2 hours. Finally, collect the cross-linked beads, and then wash the beads with DI H2O to remove residual BDDE.

After drying, the resultant cross-linked chitosan microbeads can be stored under room conditions. The content of chitosan in the wet microbeads is around 14%.

Method 2: Emulsion/Neutralization Method

### Example 4

While stirring 8.00 mL of castor oil with a small anchor paddle at 65 rpm in a 20 mL beaker, 1.00 mL of 3% Chitopharm-S in 0.15 M HCl were added drop wise, avoiding hitting the paddle. The mixture was stirred for 45 minutes and then the stir speed was increased to 100 rpm for 55 minutes. The emulsion was neutralized by addition of concentrated NH₄OH at a rate of 50 microliters/hr over 30 minutes. The emulsion was then diluted with ethanol and filtered several times to obtain the oil-free microbeads. Final washing with water and PBS, and resuspension in PBS resulted in the microbeads displayed in FIG. 4.

Cross-Linked HA Gel Phase Base for Chitosan Microbead Dermal Filler.

As noted, commercial HA-based dermal fillers such as Restylane®, Perlane®, Puragen®, the

Juvéderm® family, the Esthélis® family, and the Revanesse® family are based on cross-linking of HA with ButaneDiolDiglycidylEther (BDDE). The basis of this technology was described in Laurent (1964) and in U.S. Pat. No. 4,716,154. Briefly, HA is dissolved in a strong base, such as 1% NaOH solution, BDDE is added and the cross-linking takes place at an elevated temperature of approximately 50° C. An ether link is formed, primary at the C6 hydroxyl group. A schematic of the reaction is shown below.

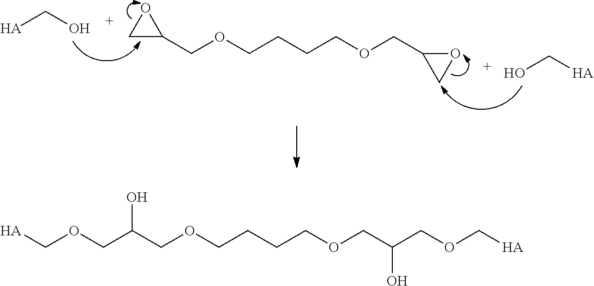

After crosslinking, the gel that is formed is collected, milled to form gel particles, and purified before filling into syringes, and terminally sterilized by moist heat. A portion of unmodified HA can be included or not, to alter somewhat the flow properties of the final product. Those skilled in the art can use this method to produce dermal fillers with excellent characteristics of biocompatibility, softness, volumizing effect, and durability.

The base continuous phase for the dermal filler system, including macroporous chitosan microbeads described herein, is the gel particle composition used in the product Revanesse® Ultra, manufactured by Prollenium Medical Technologies, Inc. Milled, purified gel particles are available by the basic process described above. At that point, prior to sterilization, a microbead component can be added to the gel phase, and mixed thoroughly, for example in a double-planetary mixer, until dispersed uniformly in the gel. This composition can then be filled into sterile syringes, prior to loading in racks and terminally sterilized by moist heat in an autoclave.

Given the size and softenss of the gel particles, and the size of the chitosan microbeads of approximately 100 μm, the overall composition can be injected into the dermis with either a 27 G or 30 G needle or cannula as demonstrated in our laboratory, and by technicians at the contract facility carrying out the rat implant study.

Biological Response—Rat Implant Study

The biological response to our filler system, is of primary importance in terms of effectiveness. This can only be determined definitively in an animal implant study or in a human clinical trial. Prior to injection into a human subject, an animal study is usually performed. In particular, as we were interested in both safety from a toxicological viewpoint, and performance in terms of tissue response and durability.

An animal study provides strong evidence for both safety and performance and to test the biological response of our chitosan bead/cross-linked hyaluronic acid filler system, we conducted a study in Sprague Dawley rats (Rattus norvegicus). This year-long study was conducted by Toxikon Corporation, a preclinical CRO in Bedford, Mass. from Dec. 31 2013 to Dec. 30 2014. Chitosan beads from our solvent evaporation method, with two different mass densities of swelled chitosan microbeads were used, as well as a sample of medium-density beads cross-linked with BDDE. These bead samples were combined with BDDE-cross-linked gel particles from a regular production lot of Revanesse Ultra and homogenized. Microbead concentrations were 25 mg/mL in all cases, HA concentration was also 25 mg/mL in all cases. The syringes were terminally-sterilized in an autoclave.

Ten Sprague Dawley rats (rattus norvegicus) were selected for the study. Two animals were assigned to each of 5 time points for histopathological examination: 1, 2, 8, 26, and 52 weeks. A volume of 0.2 mL of each of the three chitosan microbead/HA dermal filler test articles (high and low density, and medium-density cross-linked microbeads), were implanted subcutaneously into the back of each animal.

Two types of staining were used for the histopathology slides, hematoxylin and eosin (H&E), and Masson's trichrome staining, to bring out different features of the response. Generally, the trichrome staining was superior, for those characteristics in which we were most interested, in particular the evidence of stimulated collagen deposition. Collagen stains blue in the trichrome system and the deposits were evident.

Results exceeded our expectations. A normal foreign-body response was noted. Even at 1 week infiltration of cells into the macroporous beads could already be detected. In addition, there was no evidence of an excessive inflammatory reaction at any of the time points, simply the normal foreign-body reaction to the implant as a whole, clear depostion of collagen around the individual microbeads, as well as signficant collagen deposition inside the macropores of the beads, due to ingress of fibroblasts into the microbead.

A large number of tissue samples were taken with photomicrographs prepared. Some of these are displayed in the Figures. Details are discussed in the Description of the Figures:

### DESCRIPTION OF THE FIGURES

FIG. 1 The ternary phase diagram for castor oil, corn oil, and light mineral oil. Two coexistence curves are shown at temperatures of 20.7° C. and 26° C. Below these curves at the respective temperature, the system exists as two distinct phases, with different compositions. Above the curves a single phase exists, and all three components are miscible in this region. Experiments indicated that working close to the coexistence curve was necessary for optimal conditions. Final samples for the animal implant study were prepared at an evaporation temperature of 26° C., with the composition labeled as ‘1’ on the diagram (10/20/20 for castor/corn/light mineral oils).

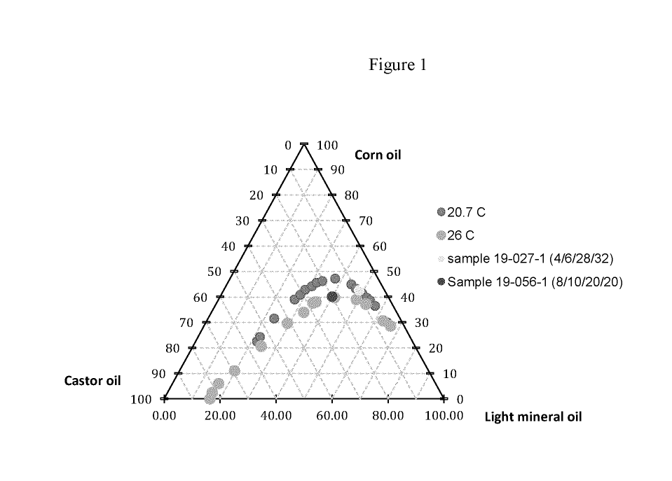

FIG. 2 Aqueous acidic chitosan droplets dispersed in castor oil in the primary emulsion. Due to the high W/O ratio some castor oil is dispersed in the aqueous droplets forming an O/W/O emulsion. This is the source of the macroporous structure of the final chitosan microbeads.

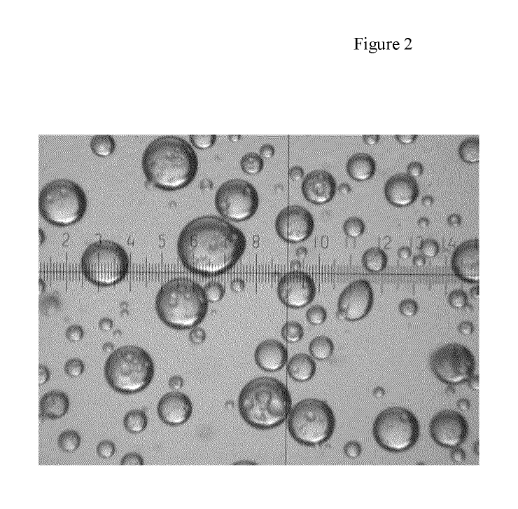

FIG. 3 Displays a photomicrograph of three samples of chitosan microbeads, produced using Method 1, emulsion/solvent evaporation. These are the same samples used in the rat implant study. The size and shape of the microbeads can be observed and in some of the beads the evidence of the macroporous structure can be seen on the surface. The size distribution (volume-weighted), mean diameter, and standard deviation around the mean are shown to the right of the photomicrograph. As can be seen the microbeads are nearly perfectly spherical. The mean diameter is ˜95 μm±20 μm. As shown in FIG. 2, by adjusting the process conditions, beads of nearly identical size can be produced with signficantly different chitosan mass densities, ranging from 9.9% to 16.2%.

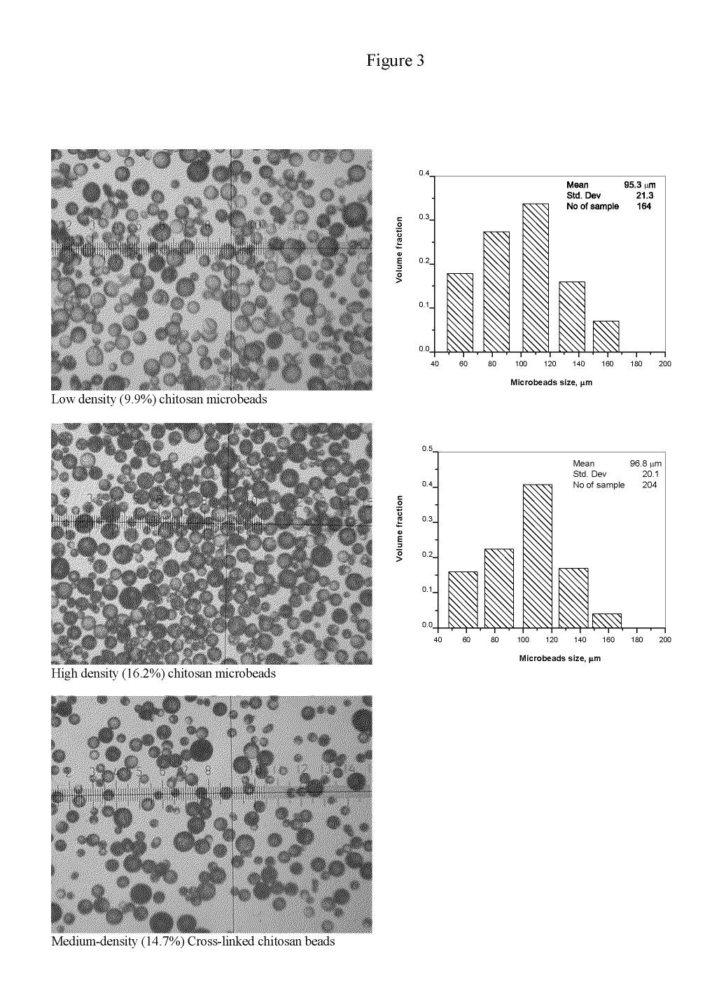

FIG. 4 Displays a photomicrograph of a sample of chitosan microbeads, produced using Method 2, emulsion/neutralization. The size distribution is again shown to the right, with a mean diamter of 98 μm±??.

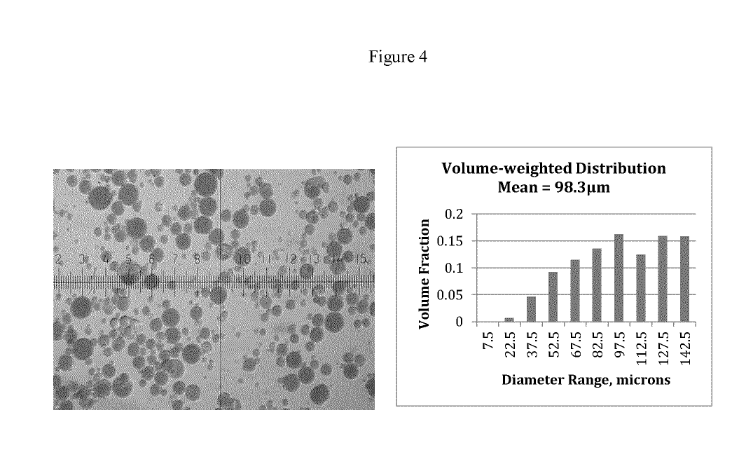

FIG. 5 Shows a result from experiments on in-vitro degradation of the dermal filler system, chitosan microbeads plus BDDE cross-linked HA gel. Bovine testicular hyaluronidase (BTH), and lysozyme were chosen as representative of enzymes that will degrade HA and chitosan respectively in mammalian systems. The storage modulus of the dermal filler gel at a frequency of 1 Hz is measured to track the degradation. As shown, neither lysozyme nor BTH alone cause rapid degradation of the filler. However, lysozyme +BTH does cause a significant decrease in the storage modulus. Finally, the base HA gel is shown to be degraded by BTH alone. This is an indication of an interaction between the HA gel and the chitosan beads. The interaction is the formation of a polyelectrolyte complex at the surface of the beads.

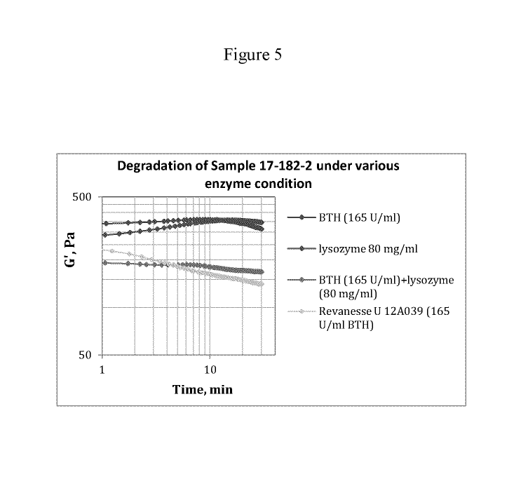

FIG. 6 A more direct demonstration of the effect that the formation of a polyelectrolyte complex has on stabilizing the chitosan microbeads against enzymatic degradation is shown here. The top two photomicrographs show chitosan beads before and after exposure to a concentrated lysozyme solution at 37° C., for 10 days in the presence of HA gel. The bottom three photomicrographs show the effect of concentrated lysozyme on the same sample of chitosan microbeads, without the presence of HA gel. After just 2 hours a bulk degradation of the microbeads is evident, and after 3 days, and finally 10 days the beads have lost most of their mass. It is seen that a rapid bulk degradation is occurring in the absence of HA.

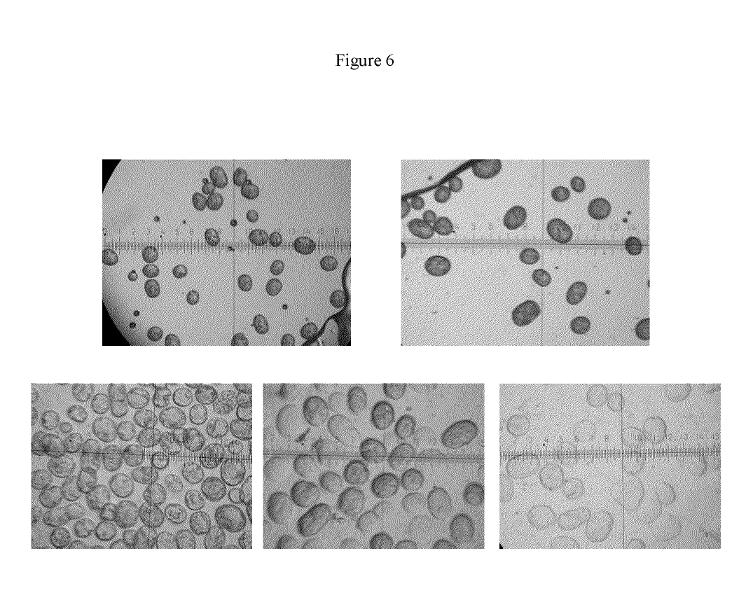

FIG. 7 First histopathology slide showing overall reaction to subcutaneous implant at 16×magnifications in the rat with low-density beads as an example. In all the histology slides shown trichrome staining was used. As noted, this brings out a feature of great interest, natural collagen depositon. On the left at 2 weeks, collagen layer below muscle is visible (blue) but very little capsule formation on bottom of implant. On right at 52 weeks, a visible thin capsule can be seen on the bottom of the implant, with no evidence of inflammation.

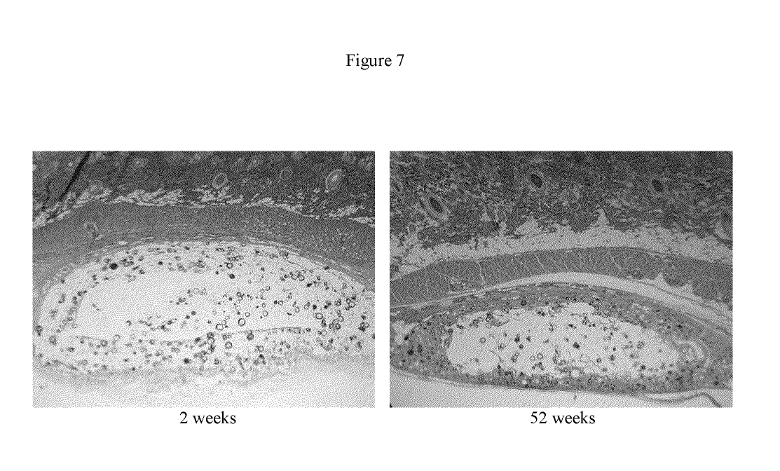

FIG. 8 At 63×magnification. These two photomicrographs demonstrate the response at 52 weeks, for cross-linked microbeads on the left and high-density microbeads on the right. In both cases there is clear evidence of collagen deposition around and on the surface of the microbeads. The high-density microbeads on the right slso show clear evidence of collagen deposition within some of the beads. This is more evident at higher magnification. Again there is no evidence of any excessive inflammatory reaction.

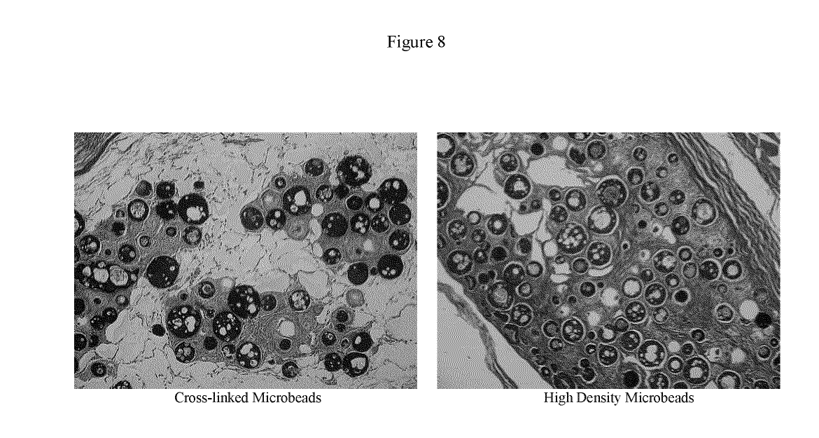

FIG. 9 In these photomicrographs at 400×magnification, evidence of collagen deposition within the microbeads is evident at 52 weeks. This is the case for low- and high-density microbeads as well as the cross-linked microbeads. Over additional time as the beads degrade the natural collagen outside and within the bead will completely occupy the space formerly taken up by the microbeads, leaving a natural collagen filling effect where the original correction took place.

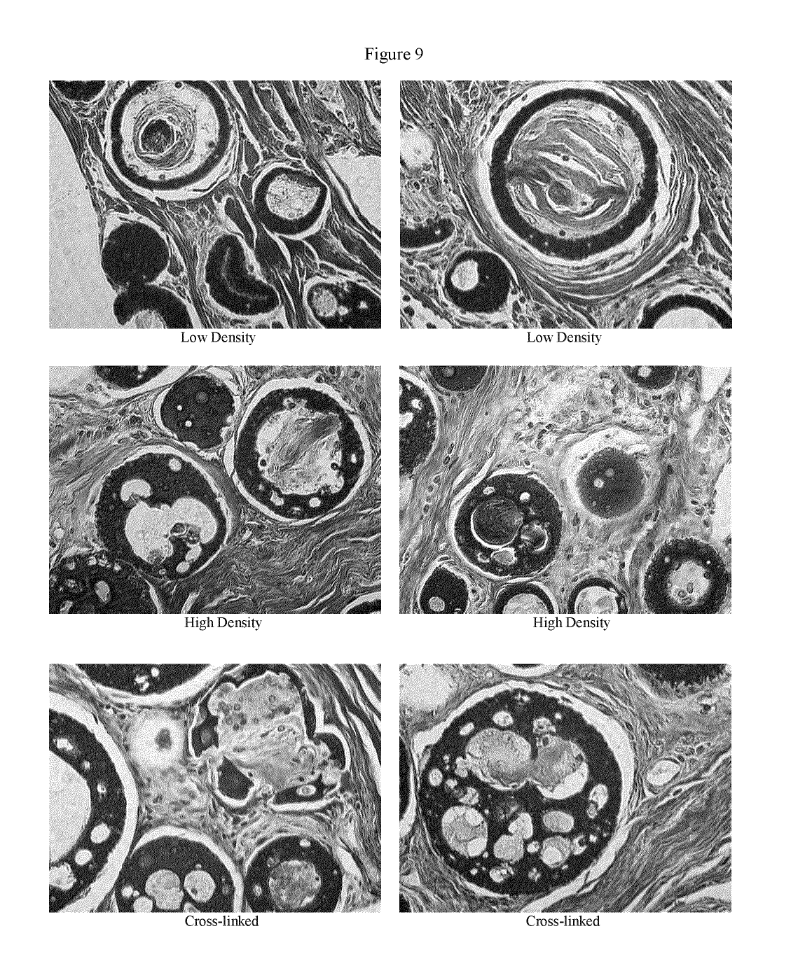

### REFERENCES

Chitosan Reviews:

K. Kurita, ‘Chemistry and application of chitin and chitosan’, Polymer Degradation and Stability, 59 (1998) 117-120

D. K. Singh and A. R. Ray, Biomedical Applications of Chitin, Chitosan, and Their Derivatives, J.M.S.—Rev. Micromole. Chem. Phys., C 40(1), 69-83 (2000)

E. Khor, ‘Review, Chitin: a biomaterial in waiting’, Current Opinion in Solid State and Materials Science 6 (2002) 313-317

M. N. V. Ravi Kumar, R. A. A. Muzzarelli, C. Muzzarelli, H. Sashiwa, and A. J. Domb, ‘Chitosan Chemistry and Pharmaceutical Perspectives’, Chem. Rev., 104, (2004) 6017-84

I. M. van der Lubben, J. C. Verhoef, G. Borchard, H. E. Junginger, ‘Review, Chitosan and its derivatives in mucosal drug and vaccine delivery’, European Journal of Pharmaceutical Sciences 14 (2001) 201-207

K. Kurita, ‘Chitin and Chitosan: Functional Biopolymers from Marine Crustaceans’, Marine Biotechnology, 8, 203-226 (2006)

R. C. F. Cheung, T. B. Ng, J. H. Wong and W. Y. Chan, ‘Review, Chitosan: An Update on Potential Biomedical and Pharmaceutical Applications’, Mar. Drugs 2015, 13, 5156-5186

I. Aranaz, M. Mengibar, R. Harris, I. Paños, B. Miralles, N. Acosta, G. Galed and A. Heras, ‘Functional Characterization of Chitin and Chitosan’, Current Chemical Biology, (2009), 3, 203-230

Chitosan Microspheres

K.C. Gupta, F. H. Jabrail, ‘Effects of degree of deacetylation and cross-linking on physical characteristics, swelling and release behavior of chitosan microspheres’, Carbohydrate Polymers 66 (2006) 43-54

A. J. Ribeiro, C. Silva, D. Ferreira, F. Veiga, ‘Chitosan-reinforced alginate microspheres obtained through the emulsification/internal gelation technique’, European Journal of Pharmaceutical Sciences 25 (2005) 31-40

V. R. Sinha, A. K. Singla, S. Wadhawan, R. Kaushik, R. Kumria, K. Bansal, S. Dhawan, ‘Review, □Chitosan microspheres as a potential carrier for drugs’, International Journal of Pharmaceutics 274 (2004) 1-33

Y. Baimark and Y. Srisuwan, ‘Preparation of Polysaccharide-Based Microspheres □ by a Water-in-Oil Emulsion Solvent Diffusion Method for Drug Carriers’, International Journal of Polymer Science, Volume 2013, Article ID 761870,6 pages

Polyelectrolyte Complexes with HA

A. Denuziere, D. Ferrier, O. Damour, A. Domard, ‘Chitosan—chondroitin sulfate and chitosan—hyaluronate polyelectrolyte complexes: biological properties’, Biomaterials, 19 (1998) 1275-1285

Y. Luo, Q. Wang, ‘Recent development of chitosan-based polyelectrolyte complexes with natural polysaccharides for drug delivery’ International Journal of Biological Macromolecules 64 (2014) 353-367

X. Deng, M. Cao, J. Zhang, K. Hu, Z. Yin, Z. Zhou, X. Xiao, Y. Yang, W. Sheng, Y. Wu, Y. Zeng, ‘Hyaluronic acid-chitosan nanoparticles for co-delivery of MiR-34a and doxorubicin in therapy against triple negative breast cancer’, Biomaterials 35 (2014) 4333-4344

T. Funakoshi, ‘Novel chitosan-based hyaluronan hybrid polymer fibers as a scaffold in ligament tissue engineering’, Journal of Biomedical Materials Research Part A, Volume 74A, Issue 3, pages 338-346 (2005)

H. Lu and N. Hu, ‘Loading Behavior of {Chitosan/Hyaluronic Acid}n Layer-by-Layer Assembly Films toward Myoglobin: An Electrochemical Study’, J. Phys. Chem. B 110, 23710-23718 (2006)

Wound Healing:

G. I. Howling , P. W. Dettmar, P. A. Goddard, F. C. Hampson, M. Dornish, E. J. Wood, ‘The effect of chitin and chitosan on the proliferation of human skin fibroblasts and keratinocytes in vitro’, Biomaterials 22 (2001) 2959-2966

K. Azuma, T. Osaki, S. Minami and Y. Okamoto, ‘Anticancer and Anti-Inflammatory Properties of Chitin and Chitosan Oligosaccharides’, J. Funct. Biomater, (2015), 6, 33-49

Biodegradability of Unmodified Chitosan

H. Kim, C. H. Tator, M. S. Shoichetl, ‘Chitosan implants in the rat spinal cord: Biocompatibility and biodegradation’, Journal of Biomedical Materials Research A, 97A(4), (2011) pp. 395-404.

J. Guzmán-Morales, C.-H. Lafantaisie-Favreau, G. Chen, C. D. Hoemann, ‘Subchondral chitosan/blood implant-guided bone plate resorption and woven bone repair is coupled to hyaline cartilage regeneration from microdrill holes in aged rabbit knees’, Osteoarthritis and Cartilage, 22 (2014) pp. 323-333

Tissue Engineering:

C. D. Hoemann, J. Sun, A. Légaré, M. D. McKee and ⊐ M. D. Buschmann, ‘Tissue engineering of cartilage using an injectable and adhesive chitosan-based cell-delivery vehicle’, OsteoArthritis and Cartilage (2005) 13, 318-329

In-Yong Kim, Seog-Jin Seo, Hyun-Seuk Moon, Mi-Kyong Yoo, In-Young Park, Bom-Chol Kim, Chong-Su Cho, ‘Research review paper ␣ Chitosan and its derivatives for tissue engineering applications’, Biotechnology Advances 26 (2008) 1-21

H. Tana, C. R. Chub, K. A. Payneb, K. G. Marra, ‘Injectable in situ forming biodegradable chitosan-hyaluronic acid based hydrogels for cartilage tissue engineering’, Biomaterials 30 (2009) 2499-2506

A. Anithaa, S. Sowmya, P. T. S. Kumara, S. Deepthia, K. P. Chennazhia, H. Ehrlich, M. Tsurkanc, R. Jayakumara, ‘Chitin and chitosan in selected biomedical applications’, Progress in Polymer Science 39 (2014) 1644-1667

L. Rami, S. Malaise, S. Delmond, J-C Fricain, R. Siadous, S. Schlaubitz, E. Laurichesse, J. Amédée, A. Montembault, L. David and L. Bordenave, ‘Physicochemical modulation of chitosan-based hydrogels induces different biological responses: Interest for tissue engineering’, Journal of Biomedical Materials Research Part A, 102(10), (2014) 3666-3676

Antimicrobial (Food):

P. K. Dutta, S. Tripathi, G. K. Mehrotra, J. Dutta, ‘Review, □ Perspectives for chitosan based antimicrobial films in food applications’, Food Chemistry 114 (2009) 1173-1182

C. Qin, H. Li, Q. Xiao, Y. Liu, J. Zhu, Y. Du, ‘Water-solubility of chitosan and its antimicrobial activity’, Carbohydrate Polymers 63 (2006) 367-374

Cross-Linked HA Dermal Fillers:

T. C. Laurent, K. Hellsing, and B. Oelotte, ‘Cross-linked Gels of Hyaluronic Acid’, Acta Chem. Scand., 18(1), (1964) pp. 274-5

1-13. (canceled)

14. A method for preparing unmodified chitosan microbeads by emulsification of an acidic chitosan solution into an oil phase in a primary emulsion containing an emulsifier, comprising the steps of:
forming a primary 0/W/O emulsion,
diluting the primary emulsion with two additional oils to form a secondary emulsion, with a three-component oil phase; and
evaporating water from droplets of the chitosan solution to form solid microbeads with a macroporous structure after oil removal.

15. The method of claim 14, wherein the oil used in the primary emulsion is castor oil.

16. The method of claim 14, wherein the emulsifier is chosen from one of:
the hydrophobic Span® family, in particular Sorbitan Monopalmitate and Sorbitan Monooleate;
the hydrophilic Tween® family, particularly PEG-20 Sorbitan Isostearate; Castor oil derivatives such as PEG-40 Castor Oil, PEG-60 Hydrogenated Castor Oil, and Polyoxyl 35 Castor Oil; Glyceryl derivatives such as Glyceryl Palmitostearate, Glyceryl Oleate, Glyceryl Trioleate, and Glyceryl Laurate; the Poloxamer family of nonionic emulsifiers, in particular Poloxamer 188; Hydrogenated Soybean Lecithin or Lecithin.

17. The method of claim 16, wherein the emulsifier is Lecithin at a concentration of from 1% to 5%.

18. The method of claim 14, wherein the two additional oils used to form the secondary emulsion are corn oil and light mineral oil.

19. The method of claim 14, wherein the secondary emulsion contains castor, corn, and light mineral oil in a preferred ratio of 10/20/20.

20. The method of claim 14, wherein the chitosan microbeads are based on chitosan with a molecular weight of from 100 to 2000 kDa.

21. The method of claim 14, wherein the chitosan microbeads have a degree of deacetylation from 65 to 95%.

22. The method of claim 14, wherein the acid is chosen from acetic acid, formic acid, adipic acid, ascorbic acid or lactic acid, or dilute inorganic acids such as hydrochloric acid or phosphoric acid.

23. The method of claim 14, wherein hydrochloric acid concentrations are between 0.1N and 0.2N, Lactic acid concentrations are between 1 and 10, and Acetic acid concentrations are between 2% and 10%.

24. The method of claim 14, wherein the acidic chitosan solution consists of a chitosan concentration between 1% and 5%.

25. The method of claim 14, wherein the W/O ratio in the primary ratio is greater than 0.75.

26. The method of claim 14, wherein the evaporation temperature is between 20° C. and 40° C.

27. A cleaning, neutralization and drying process for preparing unmodified chitosin microbeads by emulsification of an acidic chitosan solution into an oil phase in a primary emulsion containing an emulsifier, said process comprising the steps of:
forming a primary 0/W/O emulsion,
diluting the primary emulsion with two additional oils to form a secondary emulsion, with a three-component oil phase;
evaporating water from droplets of the chitosan solution to form solid microbeads with a macroporous structure after oil removal;
centrifugation and isolation of the microbeads;
washing with ethyl acetate;
repeating with n-hexane, and finally ethanol;
neutralizing with saturated Na₂C0₃;
washing with DI water;
equilibration with phosphate-buffered saline, until neutral pH achieved; and
drying by dehydration with ethanol and air drying.

28. A biocompatible, degradable dermal filler system, comprising unmodified macroporous chitosan microbeads dispersed uniformly in a continuous phase composed of cross-linked hyaluronic acid gel particles and unmodified hylauronic acid, wherein the filler is loaded into pre-sterilized syringes, as part of the filler system.

29. The system of claim 28, further comprising the pre-filled syringes being terminally sterilized by moist heat in an autoclave.

30-37. (canceled)
